(19) United States
(12) Patent Application Publication
(10) Pub. No.: US 2025/0075229 A1
(43) Pub. Date: Mar. 6, 2025

(54) Genetically Reprogrammed Extracellular Matrix

(71) Applicant: Ronawk, Inc., Olathe, KS (US)

(72) Inventor: Adam J. Mellott, Parkville, MO (US)

(21) Appl. No.: 18/726,675

(22) PCT Filed: Jan. 4, 2023
(86) PCT No.: PCT/US2023/010089

§ 371 Date: Jul. 3, 2024

Related U.S. Application Data
(60) Provisional application No. 63/296,272, filed on Jan. 4, 2022.

(51) Int. Cl. C12N 15/85; C07K 7/08

(52) U.S. Cl. CPC C12N 15/85; C07K 7/08

(57) ABSTRACT

The use of artificial sequences that may be transfected into cells for the purpose of identification of the cells themselves, as well as any extracellular matrix (ECM) material tagged by a unique artificial peptide produced by the artificial sequences are provided.

### CROSS-REFERENCE TO RELATED APPLICATIONS

This application claims priority to U.S. Provisional Application No. 63/296,272, filed Jan. 4, 2022, which is expressly incorporated by reference herein in its entirety.

### TECHNICAL FIELD

Embodiments of the presently-disclosed invention relate generally to the use of artificial sequences that may be transfected into cells for the purpose of identification of the cells themselves, as well as any extracellular matrix (ECM) material tagged by a unique artificial peptide produced by the artificial sequences.

### BACKGROUND

Alterations to DNA sequences in cells may be undertaken to impart some advantageous characteristic to the cell or produce a peptide or protein. For example, cells can be transfected to express a protein of interest in cultured cells (or an animal model) through the use of a plasmid vector or mRNA. Expression of the protein in eukaryotic cells allows the recombinant protein to be produced with proper folding and post-translational modifications required for its function. In general, transfection is the process of artificially introducing, for example, nucleic acids (DNA or RNA) into cells, utilizing means other than viral infection. Such introductions of foreign nucleic acid using various chemical, biological, or physical methods can result in a change of the properties of the cell, allowing the study of gene function and protein expression in the context of the cell. In transfection, the introduced nucleic acid may exist in the cells transiently, such that it is only expressed for a limited period of time and does not replicate, or it may be stable and integrate into the genome of the recipient, replicating when the host genome replicates.

However, there remains a need in the art for genetically modified cells, which are stably transfected with one or more sequences for identification purposes, that produce a unique artificial peptide that may be used as a unique tag for identification of ECM material formed from the genetically modified cells.

### SUMMARY OF INVENTION

One or more embodiments of the invention may address one or more of the aforementioned problems. Certain embodiments according to the invention provide a transfected cell, comprising: Sequence ID No. 1, Sequence ID No. 2, or both. The present also provides a method of tagging a cell line of interest, comprising providing the cell line of interest and transfecting cells of the cell line of interest with a vector comprising Sequence ID No. 1, Sequence ID No. 2, or both. In another aspect, the present invention provides an extracellular matrix (ECM) material, comprising Sequence ID No. 3.

### BRIEF DESCRIPTION OF THE DRAWING(S)

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout, and wherein:

FIG. 1 illustrates Sequence ID No. 1;

FIG. 2 illustrates Sequence ID No. 2; and

FIG. 3 illustrates Sequence ID No. 3.

### DETAILED DESCRIPTION

The invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

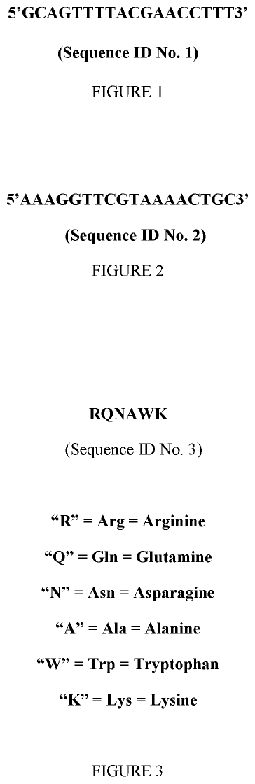

The presently-disclosed invention relates generally to the use of Sequence ID No. 1 (FIG. 1) and/or Sequence ID No. 2 (FIG. 2) for identification purposes. For example, when a novel cell line is created or a novel protein is created in a laboratory environment, the ability to identify the source from which the cell line or protein originated may be valuable. In accordance with certain embodiments of the invention, the present invention utilizes an artificial genetic sequence to stably transfect our cells of interest with so as to identify or brand the stably transfected cells that have been produced or are associated with a given source or location (e.g., in a particular laboratory) for quality control and reference purposes.

In particular, Sequence ID No. 1 (i.e., 5′GCAGTTTTACGAACCTTT3′) and its complimentary sequence, Sequence ID No. 2 (i.e., 5′AAAGGTTCGTAAAACTGC3′) may be stably transfected into cells for the purpose of identification purposes. In accordance with certain embodiments of the invention, when either sequence is successfully translated by the cell of origin, the artificial peptide sequence of Sequence ID No. 3 (i.e., RQNAWK) is present on extracellular matrices secreted by the cell or origin, where “R”-Arg-Arginine; “Q”=Gln=Glutamine; “N”=Asn=Asparagine; “A”=Ala=Alanine; “W”=Trp=Tryptophan; and “K”=Lys=Lysine. In accordance with certain embodiments of the invention, Sequence ID No. 1 and/or Sequence ID No. 2 may be embedded, for example, within collagen type 1 sequences for identification purposes. The purpose of transfecting cells with this sequence is to be able to identify all genetically modified cells that produce the unique identifier tag on collagen fibers in any acellular material that is produced. This allows an end-user to identify and cross-reference materials for associated products. For example, this identification method allows an end-user to keep track of both cells and acellular materials produced in their laboratory (or another source) for reference and cross-reference in producing new products that have been genetically modified.

Sequence ID No. 1 and Sequence ID No. 2 does not naturally exist in nature. These sequences were synthesized for producing a peptide (i.e., Sequence ID No. 3) that also does not naturally exist. This particular peptide (i.e., Sequence ID No. 3) is a tag that corresponds to the name of the filing company for the purpose of barcoding and/or branding cells that are grown with any of the filing company's platform technologies.

In accordance with certain embodiments of the invention, a variety of cells may be stably transfected with Sequence ID No. 1 and/or Sequence ID No. 2 using, for example, a non-virally vector such as lipofection, electroporation, sonication, or virally transduced using a viral vector, for example, such as adeno virus, adeno-associated virus, lenti virus or any other viral vector to deliver a plasmid construct to the nucleus of the cell host.

Accordingly, certain embodiments of the invention provide a custom molecular tag that is produced by the cells for the purpose of creating a way to track and reference both cells and acellular materials that are produced in a particular laboratory. Moreover, certain embodiments of the invention may facilitate audit production of cellular products, and to brand or tag novel cell lines or molecular products that are produced by the filing company for use.

In accordance with certain embodiments of the invention, for instance, the present invention provides a transfected cell comprising Sequence ID No. 1, Sequence ID No. 2, or both.

The transfected cell, in accordance with certain embodiments of the invention, may comprise Human Stem Cells, such as Human Wharton's Jelly Cells (MSC), Human Bone Marrow Derived Mesenchymal Stem Cells (MSC), Human Adipose Derived Mesenchymal Stem Cells (MSC), Human Skin Derived Induced Pluripotent Stem Cells (iPSC), Human Blood Cell Derived Induced Pluripotent Stem Cells (iPSCs), Human CD4+ T Cells, or Human CD8+ T Cells. Additionally or alternatively, the transfected cell may comprise Primary Mammalian Cells, such as HepG2 Cells (Liver Carcinoma Cells), Human Adult Dermal Fibroblasts (Primary Cells), Human Neonatal Dermal Fibroblasts (Primary Cells), Human Adult Keratinocytes (Primary Cells), Mouse Dorsal Root Ganglia (Primary Neural Cells), Bovine Myocytes (Primary Cell Line), Primary Porcine Hepatocytes, Porcine Chondrocytes, Porcine Osteocytes, Equine Muscle Derived Stem Cells (Primary MSCs), Primary Snail Cells, Human Macrophages, and any primary cell that can be isolated from a tissue harvested from a human, animal, or plant. Additionally or alternatively, the transfected cell may comprise Immortalized Mammalian Cell Lines, such as UB-OC2 Cells (Mouse Cochlear Epithelium), Human Myoblastoma (Muscle Tumor), PC3 (Prostate Cancer), CHO (Chinese Hamster Ovary Cell), HEK293 (Human Embryonic Kidney Cell), SHSY5Y (Neuronal Tumor), PANC-1 (Human Pancreatic Cancer), HeLa (Cervical Cancer), A549 (Lung Cancer), A673 (Muscle Cancer); and (4) Primary Plant Cells, such as Parenchymal cells, Collenchymal cells, Sclerenchymal cells, Xylem cells, Pholem cells, Meristematic cells, Epidermal cells.

In another aspect, the present invention provides a method of tagging a cell line of interest. The method may comprise providing the cell line of interest and transfecting cells of the cell line of interest with a vector comprising Sequence ID No. 1, Sequence ID No. 2, or both. In accordance with certain embodiments of the invention, the vector may comprise a non-virally vector, such as lipofection, calcium phosphate, cationic lipids, dextran, magnetofection electroporation, microinjection, ballistic transfer, optical/laser transfection, and sonication or a viral vector, such as adeno virus, adeno-associated virus, and retro virus.

In another aspect, the present invention provide an extracellular matrix (ECM) material comprising Sequence ID No. 3. In accordance with certain embodiments of the invention, the ECM material is formed from cell transfected with Sequence ID No. 1, Sequence ID No. 2, or both. The ECM, for example, may include one or more tissue components tagged with Sequence ID No. 3. For example, the one or more tissue components can comprise ECM components comprising Glycosaminoglycans (GAGs); Proteoglycans; Hyaluronic acid (HA); Heparin sulfate (HS); Chondroitin sulfate (CS); Keratan sulfate (KS); Collagen Types (e.g., Fibrillar (I, II, III, V, XI), Facit (IX, XII, XIV), Short chain (VIII, X), Basement membrane (IV), and Other (VI, VII, XIII); Elastin; DNA; RNA; Fibronectins and glycoproteins; Laminin-111 (Laminin-1); Laminin-211 (Laminin-2); Laminin-121 (Laminin-3); Laminin-221 (Laminin-4); Laminin-332/Laminin-3A32 (Laminin-5/Laminin-5A); Laminin-3B32 (Laminin-5B); Laminin-311/Laminin-3A11 (Laminin-6/Laminin-6A); Laminin-321/Laminin-3A21 (Laminin-7/Laminin-7A); Laminin-411 (Laminin-8); Laminin-421 (Laminin-9); Laminin-511 (Laminin-10); Laminin-521 (Laminin-11); Laminin-213 (Laminin-12); Laminin-423 (Laminin-14); Laminin-522; Laminin-523 (Laminin-15); Peptides; Lipids and Phospholipids; Carbohydrates; Phosphates; Saturated Fats; Unsaturated Fats; Trans Fats; Cholesterols; Organelles (e.g., Mitochondria, Smooth Endoplasmic Reticulum, Rough Endoplasmic Reticulum, Golgi body/apparatus, Lysosome, Endosome, Vesicles, Peroxisomes, Nucleolus, Nucleus, and Ribosome); and Cytoskeleton Components (e.g., Actin, Myosin).

These and other modifications and variations to the invention may be practiced by those of ordinary skill in the art without departing from the spirit and scope of the invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

That which is claimed:

1. A transfected cell, comprising: Sequence ID No. 1, Sequence ID No. 2, or both.

2. The transfected cell of claim 1, wherein the transfected cell comprises Human Stem Cells, such as Human Wharton's Jelly Cells (MSC), Human Bone Marrow Derived Mesenchymal Stem Cells (MSC), Human Adipose Derived Mesenchymal Stem Cells (MSC), Human Skin Derived Induced Pluripotent Stem Cells (iPSC), Human Blood Cell Derived Induced Pluripotent Stem Cells (iPSCs), Human CD4+ T Cells, or Human CD8+ T Cells.

3. The transfected cell of claim 1, wherein the transfected cell comprises Primary Mammalian Cells, such as HepG2 Cells (Liver Carcinoma Cells), Human Adult Dermal Fibroblasts (Primary Cells), Human Neonatal Dermal Fibroblasts (Primary Cells), Human Adult Keratinocytes (Primary Cells), Mouse Dorsal Root Ganglia (Primary Neural Cells), Bovine Myocytes (Primary Cell Line), Primary Porcine Hepatocytes, Porcine Chondrocytes, Porcine Osteocytes, Equine Muscle Derived Stem Cells (Primary MSCs), Primary Snail Cells, Human Macrophages, or any primary cell that can be isolated from a tissue harvested from a human, animal, or plant.

4. The transfected cell of claim 1, wherein the transfected cell comprises Immortalized Mammalian Cell Lines, such as UB-OC2 Cells (Mouse Cochlear Epithelium), Human Myoblastoma (Muscle Tumor), PC3 (Prostate Cancer), CHO (Chinese Hamster Ovary Cell), HEK293 (Human Embryonic Kidney Cell), SHSY5Y (Neuronal Tumor), PANC-1 (Human Pancreatic Cancer), HeLa (Cervical Cancer), A549 (Lung Cancer), A673 (Muscle Cancer); and (4) Primary Plant Cells, such as Parenchymal cells, Collenchymal cells, Sclerenchymal cells, Xylem cells, Pholem cells, Meristematic cells, Epidermal cells.

5. A method of tagging a cell line of interest, comprising: providing the cell line of interest and transfecting cells of the cell line of interest with a vector comprising Sequence ID No. 1, Sequence ID No. 2, or both.

6. The method of claim 5, wherein the vector comprises a non-virally vector, such as lipofection, calcium phosphate, cationic lipids, dextran, magnetofection electroporation, microinjection, ballistic transfer, optical/laser transfection, and sonication.

7. The method of claim 5, wherein the vector comprises a viral vector, such as adeno virus, adeno-associated virus, and retro virus, such as Lentivirus, HIV-1.

8. An extracellular matrix (ECM) material, comprising Sequence ID No. 3.

9. The ECM material of claim 5, wherein the ECM includes one or more tissue components tagged with Sequence ID No. 3.

10. The ECM of claim 9, wherein the one or more tissue components comprise Glycosaminoglycans (GAGs); Proteoglycans; Hyaluronic acid (HA); Heparin sulfate (HS); Chondroitin sulfate (CS); Keratan sulfate (KS); Collagen Types (e.g., Fibrillar (I, II, III, V, XI), Facit (IX, XII, XIV), Short chain (VIII, X), Basement membrane (IV), and Other (VI, VII, XIII); Elastin; DNA; RNA; Fibronectins and glycoproteins; Laminin-111 (Laminin-1); Laminin-211 (Laminin-2); Laminin-121 (Laminin-3); Laminin-221 (Laminin-4); Laminin-332/Laminin-3A32 (Laminin-5/Laminin-5A); Laminin-3B32 (Laminin-5B); Laminin-311/Laminin-3A11 (Laminin-6/Laminin-6A); Laminin-321/Laminin-3A21 (Laminin-7/Laminin-7A); Laminin-411 (Laminin-8); Laminin-421 (Laminin-9); Laminin-511 (Laminin-10); Laminin-521 (Laminin-11); Laminin-213 (Laminin-12); Laminin-423 (Laminin-14); Laminin-522; Laminin-523 (Laminin-15); Peptides; Lipids and Phospholipids; Carbohydrates; Phosphates; Saturated Fats; Unsaturated Fats; Trans Fats; Cholesterols; Organelles (e.g., Mitochondria, Smooth Endoplasmic Reticulum, Rough Endoplasmic Reticulum, Golgi body/apparatus, Lysosome, Endosome, Vesicles, Peroxisomes, Nucleolus, Nucleus, and Ribosome), and Cytoskeleton Components (e.g., Actin).
